(19) United States
(12) Patent Application Publication
(10) Pub. No.: US 2002/0177169 A1
(43) Pub. Date: Nov. 28, 2002

(54) Method for the preparation of a coded chemical library

(75) Inventors: Anthony Robert Corless, Hayes (GB); David Andrew Wenn, Hayes (GB); Andrew John Garman, Macclesfield (GB)

Correspondence Address: FINNEGAN, HENDERSON, FARABOW, GARRETT &, DUNNER LLP, 1300 I STREET, NW, WASHINGTON, DC, 20005, US

(73) Assignee: Zeneca Limited

(21) Appl. No.: 10/141,833

(22) Filed: May 8, 2002

Related U.S. Application Data
(63) Continuation of application No. 09/402,927, filed on Feb. 23, 2000, now abandoned.
Application No. 09/402,927 is a 371 of international application No. PCT/GB98/01077, filed on Apr. 14, 1998.

(30) Foreign Application Priority Data
Apr. 17, 1997 (GB) 9707742.4

(51) Int. Cl. G01N 33/53; G01N 33/543

(52) U.S. Cl. 435/7.1; 436/518; 436/523

(57) ABSTRACT

A method for the preparation of a coded chemical library, which method comprises synthesising the chemical library on a plurality of synthesis particles and writing code on the synthesis particles using high-energy radiation during library synthesis, so as to provide the chemical library on a plurality of coded synthesis particles, and wherein the identity of library compounds associated with a synthesis particle is established by reference to its code.

Chemical libraries are a powerful way of providing compounds for the identification of active compounds in pharmaceutical, agrochemical and related industries.

Synthesis of the compounds on beads is a prefered method since it allows generation of diversity by the “split synthesis” method (Furka A., Abstr. 14th Int. Congr. Biochem., Prague, Czechoslovakia, 1988, 5, 47; Int. J. Pept. Prot. Res, 1991, 37, 487-493) and beads are convenient reaction supports which may be sequentially exposed to different reagents and washed with relative ease. Considerable research effort has taken place to find a reliable and simple method for the identification of active compounds from a library generated on beads. One promising approach has been to “tag” the bead at various stages of the synthesis, where each tag, or component of the tag, indicates the reagent(s) to which the bead has been exposed. After testing, the tags associated with active compounds are read and the chemical structure of the active compound is inferred. A particularly useful approach is the use of mixtures of halogenated aromatic compounds, incorporated in trace amounts at each stage of the synthesis, to form an identifiable (by gas chromatography) ‘binary code’ system for ligand definition (Borchardt and Still, J. Am. Chem. Soc., 1994, 116, 373-374).

However, chemical tags have certain limitations. For example, they can limit the choice of chemistry used to construct the library. Also, chemical tags can take significant amounts of time to read.

We now disclose a method for the construction of a chemical library which avoids these problems. This method uses a laser to inscribe a code onto a bead or synthesis particle.

In a first aspect of the present invention we disclose a method for the preparation of a coded chemical library, which method comprises synthesising the chemical library on a plurality of synthesis particles and writing code on the synthesis particles using high-energy radiation during library synthesis, so as to provide the chemical library on a plurality of coded synthesis particles, and wherein the identity of library compounds associated with a synthesis particle is established by reference to its code.

By “during library synthesis” we mean in association with one or more library synthesis stages. The code indicates the chemical reaction(s) to which the synthesis particles are to be, or have been subjected. A convenient advantage of the method of the invention is that initially all the particles are essentially the same which provides significant cost advantages. It may also mean that commercially available beads may be used. We have surprisingly found that code may be written to non-rigid (eg. swellable) particles synthesis particles comprising polymers, for example polystyrene, grafted copolymers and derivatised versions thereof, and that the integrity of the code survives the rigours of library synthesis. Such rigours include the expansion and contraction of the synthesis particles.

In a preferred embodiment, the code which is written at any stage is uniquely identified with a particular chemical process, or series of chemical processes, which result in the attachment of a particular structural feature to the library compounds. Hence the set of codes written to a synthesis particle is directly indicative of the chemical compound attached to the bead.

Library synthesis is conveniently effected by the so-called split-synthesis, split-and-mix or one-compound-one-bead process originally described by Furka et al (Abstr. 14th Int. Congr. Biochem., Prague, Czechoslovakia, 1988, 5, 47) and further exemplified by Lam et al (Nature, 1991, 354, 82-84). In summary, this process involves dividing or ‘splitting’ a pool of solid-support particles, for example resin beads, into separate vessels, then reacting the particles in each vessel each with a different reagent or building block, allowing the reactions to proceed to completion, then ‘mixing’ the particles from each vessel to generate a second pool of particles which is split, reacted and mixed in the same way as above. The consequence of this process, which does not involve the use of mixtures of building block reagents, is that only one compound appears on any one particular synthesis particle.

The invention includes methods where the distribution of beads is random or directed (for example using robotic apparatus), conveniently random.

It will be understood that alternative library methods may be used which result in more than one compound per synthesis particle. The number of compounds per particle is limited only by practical considerations. In general not more than about 100 compounds are on each bead. Conveniently, not more than about 20 compounds, more conveniently not more than about 10 compounds, are on each synthesis particle.

The high energy radiation is conveniently supplied by a pulsed laser. Preferably the laser souce is selected to have a wavelength such that the radiation is absorbed by the bead material within a short distance of the surface and material is removed by the process of ablation. Convenient sources of radiation are for example excimer lasers operating at various wavelengths including 193 nm and 248 nn, Nd:YAG lasers used in conjunction with a so-called frequency-tripling or frequency quadrupling system and copper vapour lasers. These sources have the further advatage that a high-energy pulse of short duration is available. so there is minimal thermal damage to the material which is not removed. Typically, such sources will operate with a pulse duration of tens of nanoseconds with a pulse repetition rate of one pulse per 50 microseconds. Several pulses of radiation may be used but preferably the particle is marked with a single pulse of radiation. Practical considerations will include the time taken to mark beads and the cost of the laser source and the associated optics.

It is noted that for marking with a single pulse, the beads may move at very high speed before there is any possibility of motion blurring of the mark. For multiple pulse marking, since the inter-pulse interval is relatively long, the beads may move only with relatively modest speed before for example the beam position must track with the bead or motion-blurring will occur. For example, motion blurring may be said to occur if the bead moves than 1 micrometre during the marking. For a single pulse of 20 nanosecond duration the bead can have a speed of up to 50 metres per second before motion-blurring occurs. However, if 11 pulses are used then the time interval between the first and the last may be about 500 microseconds. In which case the bead speed must be less than about 2 millimetres per second. We note that even this low speed still allows several beads of typical diameter about 350 micrometres, per second to move steadily past the marking head.

The high energy radiation may be applied for example using a mask so as to apply spatially resolved radiation to each bead. The pattern on the mask is indicative of the particular chemical process or processes, for example a 30×30×30 synthesis needs 90 (30+30+30) masks.

Code may be written onto synthesis particles individually or in bulk. By individually we mean that marking is effected one particle at a time, for example by picking a particle with for example a robot and placing the particle in a writing station, or by allowing the particles to flow past a writing station. By bulk we mean for example spread out in a 2-dimensional spread. Conveniently, the particles are scattered randomly, or in a convenient formation, onto a flat surface. This allows the laser to scan the surface and access a large number of beads in a convenient time period.

Conveniently, a support such as a tray or plate, comprising a plurality of formations such as wells or indentations is used to locate the synthesis particles prior to laser treatment. Such articles may be manufactured using microengineering, and/or moulding techniques well known in the art. The wells or indentations are preferably designed so that the beads are readily located at individual loci, with no more than one bead located in each well or indentation. The laser may then scan the synthesis particles, or the particles are moved relative to the laser. The laser pulse(s) are then applied for example as the bead centre becomes aligned to the optical path of the laser. If required a recognition system may be provided so as to recognise either beads spread over a surface or beads disposed in for example wells. The disposition of the laser beam and beads is controlled using for example a controlled stage and focus arrangement, whereby to write code on individual beads.

Where code is written on a synthesis particle more than once, this is preferably written at a different location on the particle. The surface area of the synthesis particle occupied by code is conveniently of the order of about one per cent, or about 0.2-5 per cent of the total surface area. Preferred code dimensions for particular sizes of bead may be determined by routine experimentation so as to maximise the available depth of focus.

Code is conveniently read by an optical recognition system, such as an optical microscope. It is either read on-line using a camera or similar equipment, or off-line for example on a microscope slide.

In a further aspect of the invention we provide a chemical library synthesis particle comprising a code which has been applied using high energy radiation and which code may subsequently be used to identify the particle. We further provide a set of such particles. The set may comprise any convenient number of particles such as up to 10⁸, 10⁷, 10⁶, 10⁵, 10⁴, 10³, or 10² particles.

In a still further aspect of the invention we provide a coded chemical library which comprises a plurality of coded synthesis particles, each particle comprising a code which has been applied using high-energy radiation and each particle having at least one member of the library attached thereto.

The coded synthesis particles of the invention are conveniently manipulated using robotic apparatus as disclosed in our co-pending UK patent application No. 9707743.2, filed Apr. 17, 1997, the contents of which are incorporated herein by reference. Advantages of the use of manipulative robotic devices such as “pick-and-place” machines in combinatorial chemistry include: the ability to form an essentially complete library consisting of a single composite synthesis particle per chemistry either by selection from a larger stochastically formed set or by manipulation of particles at all stages; the ability to select a sub-library of controlled diversity for an initial screen which is designed to highlight the ‘volumes of chemical space’ in which compounds of interest are to be found, in particular the ability to decide not to select individual particles or sub-libraries of particles—followed by a subsequent selection of further sub-libraries surrounding the regions of interest, without further chemical synthesis processes being required. In this way, the technique significantly enhances the throughput of the overall drug discovery process.

The chemical library may be used in screening methods to identify compounds which modulate the activity of a biological of interest. Typically a compound will be cleaved from its associated coded particle before testing; alternatively, compounds are tested whilst still attached to their particles. In both cases, there needs to be an association between the measured activity and the particle that gave rise to that activity. Once a particle of interest is identified, its codes are read. In a preferred embodiment the code(s) marked on the particle are themselves directly indicative of the chemical process history. For example a code such as 12-02-09 means than the chemistry is A₁₂-B₀₂-C₀₉. This is not particle number 12-02-09, rather this is the particle that underwent chemistry processes A, B and C. Alternatively, code may be read at any convenient time such as before, during and/or after chemical synthesis.

Library compounds may be tested in a variety of assay systems. These include biochemical and in vitro assays. In general particle codes are read once the assay is complete and only beads associated with active compounds are identified and decoded. Alternatively library compounds are plated out and decoded before the assay takes place. Active compounds are then identified by reference to their position on the plate(s), for example by reference to plate number and well/locus number.

Convenient assay approaches include the following. The library may be stored with compounds removed from the synthesis particles or with compounds retained on the synthesis particles, for example in wells in microtitre plates. The library may be provided as stock solutions. These solutions may be used for many different assays. When activity is detected in an assay, the location of the active compound is determined and the corresponding synthesis particle is retrieved and decoded. Alternatively the synthesis particles are distributed in a two-dimensional assay system, for example a high density array such as on a gel or in microwells. Compounds are removed from the particles. Any active compounds give rise to zones of activity. Such an approach is disclosed for example in our zone screening patent (UK patent no. 2291708—Zeneca Limited). Corresponding particles may be decoded, either in situ or by retrieval and transferral to a code reading station.

The synthesis particle is preferably spheroid in shape, for example a bead, and comprises or contains a porous synthesis support suitably derivatised for the assembly of the compounds. Several such supports are known in the art. Particularly preferred are rigid, porous supports such as cross-linked polystyrene. We have found the following beads to be particularly useful: higher cross-linked (5%) polystyrene, Tentagel™, and CRB33 resin. A particular advantage of the methods of the present invention is that commercially available beads can be used.

Convenient sizes of synthesis particles include those of about 50-500 microns, such as 300 microns diameter. On this size of particle an individual code is conveniently of about 45 microns in length and and comprises marks of about 2-5 microns in diameter. The particles are preferably of uniform size, fine size tolerance is conveniently achieved by sieving or a similar procedure.

Under the rigours of chemical library synthesis, the synthesis particles may swell and then revert to their original size. We have found that synthesis particles may swell up to about eight times their original size under certain conditions without loss of code integrity.

In the present embodiment a preferred form of a code mark is a series of dots which are marked at a fixed pitch thereby defining the “clock” for the code against which the position of other dots is compared. A series of dots which contain the actual data to be coded, and further dots, or suitable variation in the position of one or more of the dots comprising the “clock” are conveniently provided to supply an orientation to the code. Optionally, the code may also comprise further dots which are used to supply parity or other information to allow the code to be verified, and optionally, sufficient such information to allow errors in the particular code mark to be compensated allowing reconstruction of data from a damaged code mark. These and further embodiments will be apparent to the artisan of ordinary skill.

In a further aspect of the invention, we disclose a synthesis particle which comprises a synthesis support attached to a writing surface for the purpose of carrying out the method of the invention. The synthesis support is as described above and is conveniently for example porous cross-linked polystyrene. The writing surface is conveniently a rigid surface that is suitable for writing a code with a laser and resistant to library chemistry processes. Such surfaces include for example, glass. The writing surface and the synthesis support may be combined in a variety of different shapes and fashions. We disclose two major types, heterogeneous and pseudo-homogeneous.

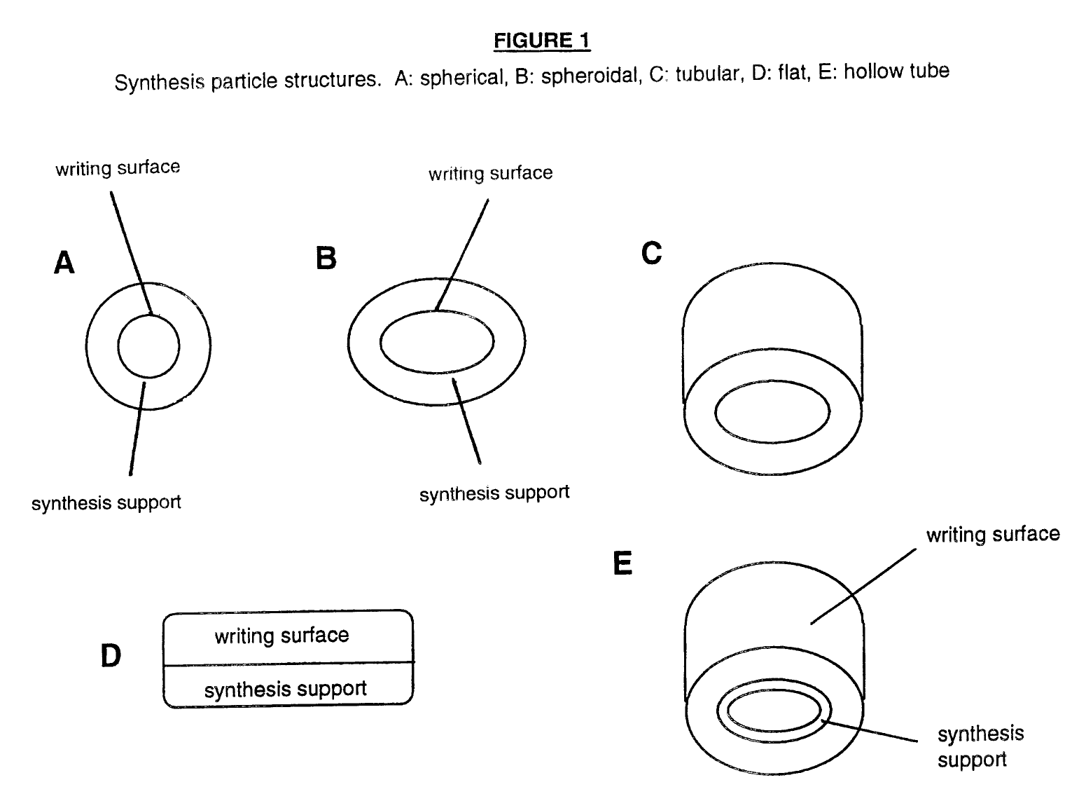

In the heterogeneous synthesis particle, the synthesis support is attached to the writing surface to form a layered structure. Such structures may be spherical or spheroid (see FIG. 1a and 1b). They may be discoid (FIG. 1c) where the synthesis support and the writing surface may be interchangeable. They may be a layered structure (FIG. 1d). Such layered structures are conveniently flat, but may be of alternative shapes as dictated by manufacturing convenience or other parameters. Optionally the synthesis support is contained within the writing surface, for example in the form of a hollow tube (FIG. 1e). It will be appreciated that alternative structures will be apparent to the scientist of ordinary skill.

In such structures, the laser beam may penetrate the synthesis layer to access the writing surface, or alternatively, the writing surface is on the outside of the particle and the synthesis particle is contained inside. In the latter case, it is important that the synthesis support is accessible to solvents and hence the writing surface must have one or more “holes”. For example, FIG. 1e has two such holes. Alternatively, a much larger number of holes is possible. At the limit, the writing surface may be porous.

For this type of particle, it is important that the laser accesses the flat writing structure at an angle that is close to orthogonal, i.e. a degree of orientation is required. Therefore, optionally, the shape of the structures may be selected such that unfavourable orientations cannot happen or are rare. For example, the tube structures of FIG. 1c and 1e may have non-orthogonal ends so that, if writing is achieved by spreading the particles out on a flat surface, the tubes will lie with the cylindrical symmetry axis parallel to the flat surface. If writing is achieved in a flow situation, an elongated or lozenge-shaped particle may conveniently provide the required degree of orientation.

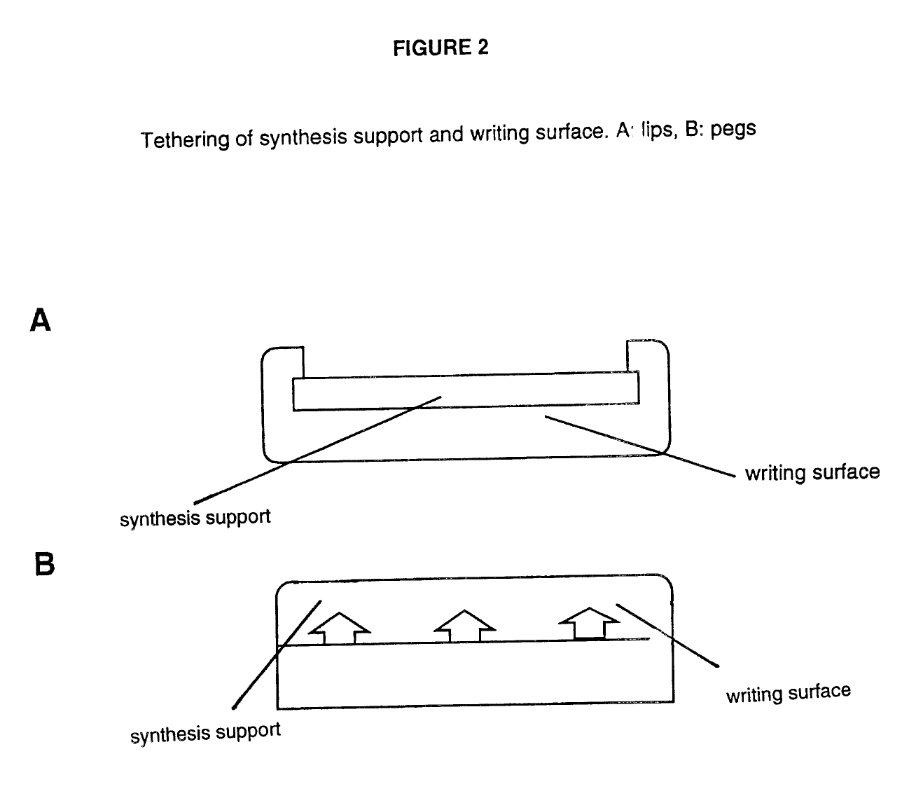

The heterogeneous particle may also contain features that ensure that the two layers do not come apart during the process. Such features may be for example lips (FIG. 2a) or pegs (FIG. 2b).

Where appropriate, the synthesis support and the writing surface may be fabricated with a support structure, for example a support layer. This support structure may provide rigidity, a means of adhering the synthesis support to the writing surface, or may facilitate manufacture.

In a further aspect of the invention we provide a pseudohomogeneous particle for use in the methods of the invention. This comprises an assembly of synthesis material and writing material such the code is written on the synthesis and/or writing material. In this type of particle the writing material contributes structural integrity. The pseudo-homogeneous particle is for example a macro-porous structure, for example a bead, in the macro-pores of which is contained a (micro) porous synthesis support. Preferably, the macro-porous structure is a controlled-pore glass bead. The (micro) porous synthesis support is preferably polystyrene, a grafted copolymer or a derivatised version thereof. Polymerisation is conveniently effected under swelling conditions.

It is common in split synthesis procedures not to mix the particles at the end of the synthesis ie after the final synthetic step. In this way information concerning the last building blocks to be added is retained. This approach may optionally be used in the methods of this invention.

Synthesis of chemical libraries of the invention may comprise any convenient number of individual reaction steps. This number is limited only by practical considerations. For example there may be up to 10, 9, 8, 7, 6, 5, 4, 3, or 2 reaction steps. At present there are typically about 2-4 reaction steps, such as 3 reaction steps.

The chemical libraries may comprise any convenient number of individual members, for example tens to hundreds to thousands to millions etc., of suitable compounds, for example peptides, peptoids and other oligomeric compounds (cyclic or linear), and template-based smaller molecules, for example benzodiazepines, hydantoins, biaryls, carbacyclic and polycyclic compounds (eg. naphthalenes, phenothiazines, acridines, steroids etc.), carbohydrate and amino acids derivatives, dihydropyridines, benzhydryls and heterocycles (eg. triazines, indoles, thiazolidines etc.). The numbers quoted and the types of compounds listed are illustrative, but not limiting.

Preferred compounds are chemical compounds of low molecular weight and potential therapeutic agents. They are for example of less than about 1000 daltons, such as less than 800, 600 or 400 daltons.

Any convenient biological of interest such as a receptor, enzyme or the like may be contacted with the chemical library as above in an assay or test system apparent to the scientist of ordinary skill.

The invention will now be illustrated but not limited by reference to the following non-limiting Figures wherein:

FIG. 1 shows synthesis particle structures comprising a synthesis support and a writing surface. In particular FIG. 1a shows spherical structures, FIG. 1b shows spheroid structures, FIG. 1c shows discoid structures, FIG. 1d shows flat layered structures and FIG. 1e shows hollow tube structures.

FIG. 2 shows two ways in which the synthesis support and writing surfaces of a synthesis particle way be tethered. FIG. 2a shows the use of lips and FIG. 2b shows the use of pegs.

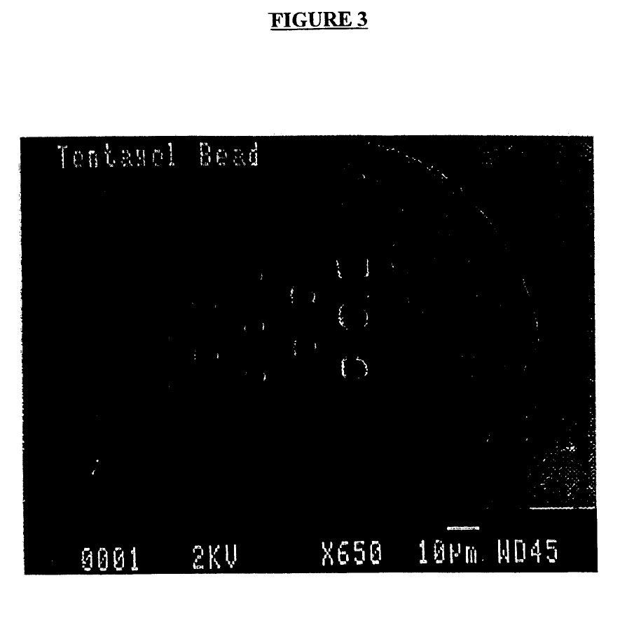

FIG. 3 shows Tentagel™ beads with code marked by laser

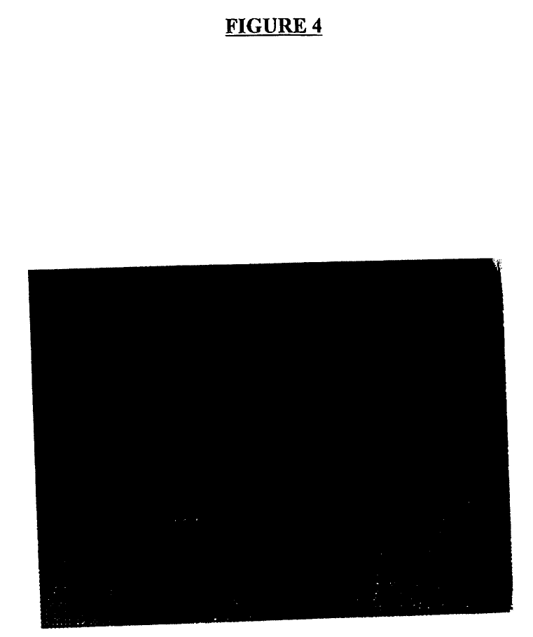

FIG. 4 shows a plurality of Tentagel™ beads with code marked by laser

1. A coded chemical library synthesis particle with an individual code applied by high energy radiation.

2. A synthesis particle as claimed in claim 1 wherein different parts of the individual code are applied at different times during the chemical library synthesis.

3. A synthesis particle as claimed in claim 2 wherein the different parts of the individual code are applied at different locations on the particle.

4. A synthesis particle as claimed in claim 1 wherein code is applied using a pulsed laser.

5. A synthesis particle as claimed in any one of the previous claims wherein different parts of the code are applied by single pulses of radiation.

6. A synthesis particle according to any one of the previous claims and being a resin bead comprising a synthesis support attached to a writing surface.

7. A pseudohomogeneous chemical library synthesis particle comprising writing and synthesis materials.

8. A coded chemical library which comprises a plurality of coded synthesis particles as claimed in any one of the previous claims, each particle having one or more members of the library attached to it.

9. A library as claimed in claim 8 wherein each particle has only one member of the library attached to it.

10. The use of a coded chemical library as claimed in claim 8 or claim 9 in screening methods to identify compounds which modulate the activity of a biological of interest.

11. A method for the preparation of a coded chemical library, which method comprises synthesising the chemical library on a plurality of synthesis particles and writing code on the synthesis particles using high-energy radiation during library synthesis, so as to provide the chemical library on a plurality of coded synthesis particles, and wherein the identity of library compounds associated with a synthesis particle is established by reference to its code.

12. The use of a pulsed laser to write a code onto a chemical library synthesis particle.

13. The use of a pulsed laser to write code onto a plurality of chemical synthesis particles in rapid succession.

14. A synthesis particle library or method for its preparation according to any one of the previous claims and wherein the code is provided by a series of dots which are marked at a fixed pitch on the synthesis particle(s).

15. The use of a robotic “pick and place” machine to manipulate synthesis particles according to any preceeding claim.
